(19) United States
(12) Patent Application Publication
(10) Pub. No.: US 2009/0155197 A1
(43) Pub. Date: Jun. 18, 2009

(54) Goniochromic indicator for skin sealant

(76) Inventors: Molly K. Smith, Atlanta, GA (US); John Gavin MacDonald, Decatur, GA (US); Bao Trong Do, Decatur, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/002,110

(22) Filed: Dec. 14, 2007

(51) Int. Cl. A61K 8/18; A61Q 1/02

(52) U.S. Cl. 424/63

(57) ABSTRACT

A skin sealant composition having a film former like cyanoacrylate and a goniochromic dye or pigment is provided. The addition of mica/metal oxide powders to the sealant formulation makes it possible to see where it has been applied, due to the mica/metal oxide's goniochromic (e.g. irridescent or pearlescent) appearance, and it is also still possible to view the underlying skin, especially from an angle at which the color is less apparent. Thus, mica/titanium dioxide or other goniochromic or light interfering particles may be used as an additive to allow for determination of complete coverage, while not significantly obscuring the incision site.

### BACKGROUND OF THE INVENTION

Surgical site infections (SSI) occur following about 2-3 percent of surgeries in the United States with an estimated 500,000 incidents of SSI occurring annually, which can lead to significant patient morbidity and mortality. In addition to the negative impact of such infections on patient health, these potentially avoidable infections contribute significantly to the financial burden experienced by the health care system. SSIs result when an incision becomes contaminated by bacteria, and for most surgeries the primary source of these infection-causing microorganisms is the skin (an exception being surgeries in which the gastrointestinal tract is penetrated).

Various compositions are used to prepare the skin prior to surgery. Skin preparations or “preps” are used to remove some level of microbial load on the skin prior to making an incision. Skin sealant materials are used to protect patients from bacterial infections associated with surgical site incisions and insertion of intravenous needles. Skin preps are applied to the skin and allowed to dry to maximize effectiveness for reducing microorganisms. After the skin prep has dried, the sealant may be applied directly to the skin in liquid form. The sealant forms a coherent film with strong adhesion to the skin through various techniques based on the chemistry of the sealant.

Skin preps currently are predominantly povidone-iodine or chlorhexidine gluconate based formulations and may contain alcohol for fast drying and more effective killing of organisms. Time constraints in the operating room and the lack of an indicator that the prep has dried often result in the skin remaining wet when draping and/or surgery begin, creating the possibility of infection. The lack of an indicator can also negatively impact infection since the users cannot know with certainty where the prep and sealant have been applied.

Skin sealants now use a polymer composition that dries to form a film through evaporation of a solvent, for example. Other skin sealants contain monomeric units that polymerize in situ to from a polymeric film. Cyanoacrylate sealants containing alkyl cyanoacrylate monomer are an example of the latter type wherein the monomer polymerizes in the presence of a polar species such as hydroxide, water or protein molecules to form an acrylic film. The resulting film serves to immobilize bacterial flora found on the skin and prevents their migration into an incision made during a surgical procedure or skin puncture associated with insertion of an intravenous needle. A thin coating is desired but in some cases, medical personnel apply multiple coats of sealant to ensure that complete coverage has been achieved. This results, however, in reduced product performance as thicker layers of sealant may become brittle and flaky.

In addition to the film former, skin sealants contain plasticizing agents to improve film flexibility and conformance. This is desirable in skin sealant applications in order to prevent the film from cracking or flaking off during use and so that the film is flexible enough to allow for movement or adjustment of the limb or appendage without compromising the barrier properties of the sealant film. They may also include viscosity modifiers to aid in application of the liquid composition, free radical and anionic scavengers to stabilize the product prior to use, biocidal agents to kill immobilized bacteria under the film, and the like.

Skin sealants have also been formulated with colorants to help the user apply the liquid composition uniformly to the skin, especially when large areas are to be covered. There are several problems, however, with existing colorants; addition of a colorant directly to the liquid skin sealant composition can negatively impact both in situ polymerization rates and the conversion reaction, in the case of cyanoacrylate compositions, or evaporation rates and the coalescence process in the case of polymer solution compositions. Lastly, after completion of the surgical procedure, the colorant in the sealant can obscure the wound site, making it difficult to detect redness associated with surgical site infections, bruising or leakage.

It is clear that there exists a need for a colorant that provides a visual cue to indicate coverage area that does not obscure the wound site.

### SUMMARY OF THE INVENTION

In response to the foregoing difficulties encountered by those of skill in the art, we have discovered that addition of mica/metal oxide powders to the formulation makes it possible to see where it has been applied, due to the mica/metal oxide's goniochromic (e.g. irridescent or pearlescent) appearance, and it is also still possible to view the underlying skin, especially from an angle at which the color is less apparent. Thus, mica/titanium dioxide or other goniochromic or light interfering particles may be used as an additive to allow for determination of complete coverage, while not significantly obscuring the incision site.

### DETAILED DESCRIPTION OF THE INVENTION

Skin preparations or “preps” are used to remove some level of microbial load on the skin prior to making an incision. Skin preps are applied to the skin and allowed to dry to maximize effectiveness for reducing microorganisms. Skin preps currently are predominantly povidone-iodine or chlorhexidine gluconate based formulations and may contain alcohol for fast drying and more effective killing of organisms. Povidone iodine, available commercially as Betadine® is estimated to be used in 80 percent of surgeries as a skin preparation. Betadine® skin prep is an aqueous solution of 10 percent povidone iodine having 1 percent titratable iodine content. When Betadine® skin prep is applied to the skin, it imparts and orange-brown color. Chloraprep® skin prep is another common formulation and is chlorhexidine gluconate based.

Skin sealant materials are used to protect patients from bacterial infections associated with surgical site incisions and insertion of intravenous needles. Skin sealants are often applied directly over or on top of skin preps. The sealant forms a coherent film with strong adhesion to the skin through various techniques based on the chemistry of the sealant composition.

The skin sealants used herein contain a film former and a plasticizer and other optional ingredients like viscosity modifiers to aid in application of the liquid composition, free radical and anionic scavengers to stabilize the product prior to use, biocidal agents to kill immobilized bacteria under the film, and the like.

One film former available in a skin sealant composition is commercially known as InteguSeal® and is available from Medlogic Global, Ltd of Plymouth, England. InteguSeal® skin sealant contains medical grade n-butyl cyanoacrylate monomer (80% w/w). Medical grade cyanoacrylate is double distilled. Non-medical grade cyanoacrylate, in contrast, is single distilled and is typically marketed as a “super glue” type adhesive for gluing a wide variety of substrates together.

Applying the sealant to the skin may involve the use of dispensers developed for that purpose. One exemplary dispenser has the liquid sealant held in at least one oblong glass ampoule within a rigid nylon housing. The housing has a body and a cap that are slidably connected and it is the cap which holds the ampoule(s). In use, the two parts are moved toward each other to dispense the liquid; the cap moving into the body. Moving the parts together results in breakage of the glass ampoule(s) and dispensing of the liquid. A detent-type locking mechanism holds the body and cap together once they are moved. The locking mechanism consists of slots formed in the cap into which fits a slight protuberance or knoll of plastic formed on the inside surface of the body. Once the ampoule is broken, the liquid travels through a small piece of foam which catches any glass shards that may have been formed by the breakage of the ampoule and thence on to the tip portion of the body. The tip has a number of small holes in it to allow the liquid to pass through. The body tip has a piece of foam on the outside, held in place with a rigid plastic oval-shaped ring that snaps in place on the tip. The outer foam contacts the skin of the patient when the liquid is dispensed. Other types of dispensers may be found in U.S. Pat. Nos. 4,854,760, 4,925,327 and 5,288,159, incorporated herein by reference.

The sealant may be packaged in a “kit” form for use in medical facilities and bundled with the appropriate skin prep solution for ease of use and the convenience of the medical personnel. In addition, various complimentary or “mating” containers and different packaging schemes have been used for some time and are known in the art.

In addition to being used as a traditional skin sealant, i.e. as a plasticized film forming barrier through which a surgical incision is made, the goniochromic particle and skin sealant composition may also be used like a bandage to close and/or cover wounds, abrasions, burns, acne, blisters and other disruptions in the skin to protect them from subsequent contamination. The use of the skin sealant composition would therefore not be limited to medical personnel.

The use of plasticizer in the skin sealant is important in order to give the film enough flexibility and conformability in order prevent cracking or flaking of the coating. The range of plasticizer required is about 10 to 60% wt/wt, more particularly between 15 and 50% and most particularly between 18 and 25% wt/wt. Common plasticizers that may be used include tributyl o-acetyl citrate, acetin, other alkyl substituted citrate derivatives, dioctylphthalate and acrylic acid monomer. Parts per million (“ppm”) denotes one particle of a given substance for every 999,999 other particles. This is roughly equivalent to one drop of ink in a 150 liter (40 gallon) drum of water, or one second per 280 hours (11 days, 16 hours). One part in 10⁶—a precision of 0.0001%.

The intensity or brightness of light is expressed in lux (lx), for example, an over cast summer day is estimated to between 30,000 lx and 40,000 lx and a mid-winter day is estimated to be about 10,000 lx. The British Standards Institution Code of Practice for Day-lighting, BS 8206 Part 1 deals in general terms with the code of practice for artificial light. The following gives some general guidance for the light requirements for the work place.

General office, laboratories, kitchen—500 lx

Drawing offices—750 lx

Tool rooms and paintwork—1000 lx

Inspection of graphic reproduction—1500 lx.

Accordingly, for purposes of this disclosure “normal light conditions” refers to light conditions of between about 500 lx and 2000 lx, more desirably, from about 750 lx to about 1500 lx as determined in accordance with BS 8206 part 1.

One way to address the problem described in the introduction is to include goniochromic particles like mica/metal oxide particles in the formulation. Mica is a layered silicate, and coating mica with metal oxides provides an iridescent effect. Such mica/metal oxide particles are commonly utilized in cosmetics such as eye shadow and nail polish to provide iridescence, and both the composition and thickness of the metal oxide layer determine the background color and iridescent color of the particles. Generally speaking, the cyanoacrylate can be mixed with the mica using a stir bar or other mechanical means.

Titanium dioxide and iron oxide are the most often used metal oxides. These metal oxides are both highly refractive and reflective. When they are coated onto thin sheets of mica, they create interesting light effects that can be seen as goniochromism.

Goniochromism is derived from the greek words gonia which means angle, and chroma which means color. It is the property of certain surfaces to change their color depending on the angle under which they are viewed. From a physics standpoint this is rather uncommon behavior, and in man-made objects this is usually found in materials that are specifically designed for the effect. Examples are pearlescent and iridescent particles or interference pigments.

One way in which the goniochromism discussed herein is produced is believed to be that light is reflected at the metal oxide surface, and also bent and reflected at the boundaries between the metal oxide and the mica. In this way, the metal oxide layer reflects light twice, and the delay between the first and second reflection slightly shifts the phase of the incident light's wavelength. This shift cancels out (or interferes with) some wavelengths of light, and amplifies others. These amplified wavelengths determine the dominant iridescent color. The thickness of the metal oxide layer determines the size of the phase shift that occurs, and therefore determines the color of iridescence. For example, a 50 nm thick coating of titanium dioxide has a silver appearance, while a 100 nm thick coating of titanium dioxide appears red. The brilliance of iridescence declines for coatings thicker than 150 nm.

Commercially available goniochromic pigments include those used in the Examples below as well as those known as Helicone® pigments from LCP Technology GmbH of Burghausen, Germany, SpectraFlair® pigments from JDS Uniphase Corporation of Milpitas, Calif. and Metallyte® pigments from ExxonMobil. Mica/titanium dioxide powders have an advantage in that they are well known to be approved for cosmetic use, and some were recently approved for use in ingestible drug coatings, so they have a favorable safety profile for use in a surgical skin sealant.

It was hypothesized that creating a suspension of mica/metal oxide powder in the cyanoacrylate Integuseal® skin sealant formulation would provide a means for visualizing skin coverage due to the iridescence, while still allowing a clear view of the underlying skin since only a small amount of mica/metal oxide is needed for iridescence and some mica particles are even translucent or transparent when viewed at the proper angle. The inventors have found that viewing the coating at angles differing by about 30 degrees or more (e.g. 45 or 55 degrees) provides sufficient difference so that one view is of a color and another is a relatively unobstructed view of the underlying surface. In this manner, a first color is visible at one angle and a second color is visible at a second angle. The second color may be translucent or transparent.

Another advantageous aspect of using mica is that the goniochromism would likely be visible on skin tones ranging from light to dark, while most traditional colorants (dyes, pigments) would be difficult to see on darker skin. In practice, it would be advisable to choose a color that is dissimilar to a skin condition such as bruising, irritation, inflammation, jaundice, bleeding and the like, so that one may clearly see the extent of such a condition.

In order to test this hypothesis, a variety of cosmetic-grade mica/metal oxide powders of various colors were added to Integuseal® skin sealant and their appearance on skin was evaluated. The colors that appeared to provide the best balance of indicating where the sealant had been applied without significantly obscuring the skin tended to be metallic (e.g. gold, silver) and particularly sparkly. Also promising for this application was mica that had a very faint background color with pink-purple iridescence, as it nearly vanished when held at an angle where the iridescence was not apparent but was clearly visible when iridescent. In all of the Examples, the testing was done with the unaided human eye under normal lighting conditions.

For each color of mica/metal oxides tested, approximately 1-10 mg of the powder was placed on a glass microscope slide, 3-4 drops (60-80 mg) of colorless Integuseal® skin sealant were applied to it and then mixed using a spatula. Fifteen Integuseal® skin sealant mixtures were prepared in this manner, each containing a different mica/metal oxide to provide a variety of colors and iridescent effects. These mixtures were applied as a thin layer on a glass slide for evaluation of suitability of color, and six of them were evaluated further by applying a thin layer to the skin. This was done to determine whether it was easy to see where the sealant had been applied and whether it was possible to still observe the underlying skin. In addition, three of these six were further tested by applying them over Betadine® skin prep.

### EXAMPLE 1

On a glass microscope slide, 10 mg of “Sunray Gold” mica powder (mica coating with titanium dioxide, iron oxide. From Coastal Scents, dba K-Plex LLC, Naples, Fla.) was mixed with 4 drops of colorless Integuseal® skin sealant. A portion of this mixture was spread into a thin layer on a separate microscope slide and allowed to polymerize. A sparkly gold iridescence was observed in the polymerized coating. A small amount of this mixture was also applied to untreated skin and to skin treated with Betadine. In both cases, the gold iridescence was visible to indicate where the mixture had been applied, but at certain angles the iridescence was less apparent and the underlying skin tone and features (e.g. blood vessels) were easily observed.

### EXAMPLE 2

On a glass microscope slide, 7 mg of “All Sparkle” mica powder (from SweetScents LLC, Westminster, Colo.) was mixed with 3 drops of colorless Integuseal® skin sealant. A portion of this mixture was spread into a thin layer on a separate microscope slide and allowed to polymerize. A silvery iridescence was observed in the polymerized coating.

### EXAMPLE 3

On a glass microscope slide, 9 mg of “Diamond Cluster” mica powder (SweetScents LLC) was mixed with 4 drops of colorless Integuseal® skin sealant. A portion of this mixture was spread into a thin layer on a separate microscope slide and allowed to polymerize. A sparkly silver iridescence was observed in the polymerized coating. A small amount of this mixture was also applied to untreated skin and to skin treated with Betadine. In both cases, the silver iridescence was visible to indicate where the mixture had been applied, but at certain angles the iridescence was less apparent and the underlying skin tone and features (e.g. blood vessels) were easily observed.

### EXAMPLE 4

On a glass microscope slide, 6 mg of “Delta Sunrise” mica powder (SweetScents LLC) was mixed with 4 drops of colorless Integuseal® skin sealant. A portion of this mixture was spread into a thin layer on a separate microscope slide and allowed to polymerize. A light pinkish-purple iridescence was observed in the polymerized coating. A small amount of this mixture was also applied to untreated skin and to skin treated with Betadine. In both cases, the iridescence was visible to indicate where the mixture had been applied, but at certain angles the iridescence nearly vanished and the underlying skin tone and features (e.g. blood vessels) were easily observed.

### EXAMPLE 5

On a glass microscope slide, 7 mg of “Vanilla Sugar Cookie” mica powder (SweetScents LLC) was mixed with 3 drops of colorless Integuseal® skin sealant. A portion of this mixture was spread into a thin layer on a separate microscope slide and allowed to polymerize. A bronze/golden iridescence was observed in the polymerized coating. A small amount of this mixture was also applied to untreated skin. The bronze/golden iridescence was fairly effective in indicating where the mixture had been applied, and still allowed the underlying skin tone and features (e.g. blood vessels) to be easily observed.

### EXAMPLE 6

On a glass microscope slide, 4 mg of “White Silk SuperShine” mica powder (SweetScents LLC) was mixed with 3 drops of colorless Integuseal® skin sealant. A portion of this mixture was spread into a thin layer on a separate microscope slide and allowed to polymerize. A white, slightly silver iridescence was observed in the polymerized coating.

### EXAMPLE 7

On a glass microscope slide, 6 mg of “Xian Vistas” mica powder (mica coated with titanium dioxide and iron oxide. Coastal Scents) was mixed with 4 drops of colorless Integuseal® skin sealant. A portion of this mixture was spread into a thin layer on a separate microscope slide and allowed to polymerize. A greenish-gold iridescence was observed in the polymerized coating. A small amount of this mixture was also applied to untreated skin. The greenish-gold iridescence was fairly effective in indicating where the mixture had been applied, and still allowed the underlying skin tone and features (e.g. blood vessels) to be observed to some extent.

### EXAMPLE 8

On a glass microscope slide, 8 mg of “Cactus Sun” mica powder (mica coated with titanium dioxide, iron oxide and tin oxide. Coastal Scents) was mixed with 4 drops of colorless Integuseal® skin sealant. A portion of this mixture was spread into a thin layer on a separate microscope slide and allowed to polymerize. A green-gold iridescence was observed in the polymerized coating.

### EXAMPLE 9

On a glass microscope slide, 5 mg of “Arizona Sandstone” mica powder (SweetScents LLC) was mixed with 4 drops of colorless Integuseal® skin sealant. A portion of this mixture was spread into a thin layer on a separate microscope slide and allowed to polymerize. A darkish bronze iridescence was observed in the polymerized coating.

### EXAMPLE 10

On a glass microscope slide, 6 mg of “Evening Star” mica powder (SweetScents LLC) was mixed with 3 drops of colorless Integuseal® skin sealant. A portion of this mixture was spread into a thin layer on a separate microscope slide and allowed to polymerize. A sparkly silver iridescence was observed in the polymerized coating, with the relatively large individual mica particles visible.

### EXAMPLE 11

On a glass microscope slide, 2 mg of “Moon” mica powder (SweetScents LLC) was mixed with 3 drops of colorless Integuseal® skin sealant. A portion of this mixture was spread into a thin layer on a separate microscope slide and allowed to polymerize. A light purple iridescence was observed in the polymerized coating.

### EXAMPLE 12

On a glass microscope slide, 3 mg of “Tropical Starlight” mica powder (SweetScents LLC) was mixed with 4 drops of colorless Integuseal® skin sealant. A portion of this mixture was spread into a thin layer on a separate microscope slide and allowed to polymerize. A pinkish-coral iridescence was observed in the polymerized coating.

### EXAMPLE 13

On a glass microscope slide, 1 mg of “Bewitched” mica powder (SweetScents LLC) was mixed with 3 drops of colorless Integuseal® skin sealant. A portion of this mixture was spread into a thin layer on a separate microscope slide and allowed to polymerize. An olive-brown iridescence was observed in the polymerized coating.

### EXAMPLE 14

On a glass microscope slide, 7 mg of “Aqua” mica powder (mica coated with titanium dioxide, chromium green oxide, ferric ferrocyanide. Coastal Scents) was mixed with 4 drops of colorless Integuseal® skin sealant. A portion of this mixture was spread into a thin layer on a separate microscope slide and allowed to polymerize. An aqua blue-green iridescence was observed in the polymerized coating. A small amount of this mixture was also applied to untreated skin. The aqua iridescence was effective in indicating where the mixture had been applied, but interfered with observation of the underlying skin tone and features (e.g. blood vessels).

### EXAMPLE 15

On a glass microscope slide, 1 mg of “Amythest” mica powder (SweetScents LLC) was mixed with 3 drops of colorless Integuseal® skin sealant. A portion of this mixture was spread into a thin layer on a separate microscope slide and allowed to polymerize. A purple iridescence was observed in the polymerized coating.

These experiments illustrate that goniochromic particles like mica/metal oxide can be used as a skin sealant additive that allows the user to clearly see where they have applied the sealant, while simultaneously allowing them to view the underlying skin. The useful range was between about 1.25 and 16.7 weight percent mica/metal oxide in cyanoacrylate, more particularly between 3 and 12 weight percent and still more particularly between 5 and 10 weight percent, depending on the color. Other materials having iridescent or sparkly effects (e.g. non-mica glitters) discussed above are also promising additives for improving the visibility of skin sealant.

### EXAMPLES OF VIEWING ANGLES

The skin sealant compositions applied to microscope glass slides cited in a number of the examples above were studied to determine the viewing angle and when the color was seen and then at what angle the color vanished to yield a transparent coating. The transparent coating would allow the surgeon to observe the skin surface for the procedure to begin or observation of the wound after surgery was completed.

The coatings were observed with the unaided eye under normal lighting conditions starting from a perpendicular viewing angle (90 degree to the slide surface). The observer then tilted the slide off the 90 degree angle and stopped when the colored coating became colorless or transparent. That angle was recorded. The following table lists the angles of various coatings.

| | Angle off perpendicular |
|---|---|
| Pigment Type | (degree) |
| Delta Sunrise | 45 |
| Aqua | 45 |
| Sunray Gold | 55 |
| Cactus Sun | 30 |
| Diamond Cluster | 30 |

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A skin sealant composition comprising a film former and a goniochromic pigment.

2. The skin sealant composition of claim 1, wherein said film former is cyanoacrylate.

3. The composition of claim 1, wherein said goniochromic pigment is a mica/metal oxide is present in an amount between 1.25 and 16.7.

4. The composition of claim 1, wherein said goniochromic pigment is a mica/metal oxide is present in an amount between 3 and 12 weight percent.

5. The skin sealant composition of claim 3 wherein said mica/metal oxide is present in an amount between 5 and 10 weight percent.

6. The skin sealant composition of claim 1 used to cover wounds, abrasions, burns, acne, blisters and other disruptions in the skin to protect the skin from subsequent contamination.

7. The skin sealant of claim 1 wherein said goniochromic pigment is of a color dissimilar from a skin condition such as bruising, irritation, inflammation, jaundice, bleeding and the like.

8. The skin sealant composition of claim 1 further comprising a plasticizer selected from the group consisting of tributyl O-acetylcitrate and acetin.

9. A medical kit comprising a skin sealant, plasticizer and a goniochromic pigment.

10. The medical kit or claim 9 wherein said goniochromic pigment is a mica/metal oxide.

11. The medical kit of claim 9 further comprising an iodine-containing skin prep, a plasticizer and a mica/metal oxide powder, wherein the color is visible to an unaided human eye under normal light conditions.

12. A skin sealant composition comprising a film former and a sufficient quantity of a mica/metal oxide such that a first color is visible at one angle and a second color is visible at a second angle, to an unaided human eye under normal light conditions.

12. The skin sealant of claim 11 wherein said second color is translucent or transparent.

13. The skin sealant of claim 11 wherein said angles differ by at least 30 degrees.

14. The skin sealant of claim 11 wherein said angles differ by at least 45 degrees.
